(19) United States
(12) Patent Application Publication
(10) Pub. No.: US 2005/0239701 A1
(43) Pub. Date: Oct. 27, 2005

(54) Transient and/or permanent modification of sexual behavior and/or fertility using recombinant chimeric GnRH

(76) Inventors: Henry Baker, Birmingham, AL (US); De-chu C. Tang, Birmingham, AL (US); Kent Rigby Van Kampen, Birmingham, AL (US)

Correspondence Address: FROMMER LAWRENCE & HAUG, 745 FIFTH AVENUE- 10TH FL., NEW YORK, NY, 10151, US

(21) Appl. No.: 10/966,842

(22) Filed: Oct. 15, 2004

Related U.S. Application Data
(63) Continuation-in-part of application No. PCT/US03/11590, filed on Apr. 16, 2003.
(60) Provisional application No. 60/373,244, filed on Apr. 16, 2002.

(51) Int. Cl. A61K 48/00; A61K 38/24; A61K 38/16

(52) U.S. Cl. 514/12; 514/44; 424/93.2

(57) ABSTRACT

The invention provides an immunogenic composition comprising a GnRH multimer and an antigenic carrier, an immunogenic composition comprising a recombinant vector containing a nucleic acid molecule encoding a GnRH multimer and optionally an antigenic carrier, antibodies elicited by the immunogenic compositions, and methods of using the immunogenic compositions and antibodies for modifying sexual physiology and behavior, improving the organoleptic properties of meat, and treating androgen-dependent prostate tumors and GnRH-sensitive ovarian tumors.

### REFERENCE TO RELATED APPLICATIONS AND MATERIALS

This application is a continuation-in-part of International Patent Application PCT/US2003/011590 filed Apr. 16, 2003 and published as WO 2003/08950 on Oct. 30, 2003, which claims priority from U.S. provisional application Ser. No. 60/373,244, filed on Apr. 16, 2002. The above referenced applications, and all applications and other documents cited in the following text, and all documents cited or referenced in the documents cited in the following text, are incorporated herein by reference. Documents incorporated by reference into this text or any teachings therein may be used in the practice of this invention. Documents incorporated by reference into this text are not admitted to be prior art. Furthermore, authors or inventors on documents incorporated by reference into this text are not to be considered to be “another” or “others” as to the present inventive entity and vice versa, especially where one or more authors or inventors on documents incorporated by reference into this text are an inventor or inventors named in the present inventive entity.

### FIELD OF THE INVENTION

The present invention relates to the use of recombinant vectors containing genetic sequences encoding multimers of gonadotropin releasing hormone (GnRH) alone or in combination with genetic sequences encoding bacterial toxins such as the Clostridium tetani C toxin fragment. These recombinant vectors can be administered by various means orally, topically, mucosally, or by injection into body tissues (e.g., intradermal injection) to modify the sexual behavior or fertility, or both, of vertebrates by inducing an immune response that disorders sexual physiology.

### BACKGROUND

Accurate data on the magnitude of the pet overpopulation problem are not available, but, based on a recent survey of 40 animal control and humane agencies located across the United States, it is estimated that 10 to 15 million unwanted cats and dogs are born in the United States each year (American Pet Products Manufacturers Association, 1996). Less than 5% of these are adopted and 40% of those adopted are returned. In addition, it is estimated that there are between 40 and 60 million homeless cats. The suffering created by abandonment, abuse, mass killing and the hardships endured by homeless strays probably constitutes the single greatest source of cruelty to two of our most endeared pet species. Additionally, every community must support animal control units and shelters at an enormous financial burden. It is estimated that animal control and humane organizations spend $250 to capture, process, adopt or euthanize each dog or cat. This amounts to an national expenditure of $2.5 to 3.75 Billion each year (FIG. 1). Sincere attempts have been made to stem this tide, but the problem continues unabated and will continue until an effective non-surgical contraceptive is developed for dogs and cats (Anchel, 1990). The only direct attempt to stem this tidal wave is surgical spaying and neutering of very few adopted animals. While this effort is noble and commended as a sincere attempt against overwhelming odds, it cannot hope to affect any perceptible change. The technical complexity of this procedure, coupled with its high cost and high rate of noncompliance, doom it to failure. The ideal cat and dog contraceptive must be simple to administer, requiring no more than an injection or oral administration, capable of being given rapidly to large numbers of animals, effective in preventing conception and reproductive behavior, safe, inexpensive, and ideally linked with rabies immunization, including bait-drop based immunization, which is readily accepted by animal owners and control agencies.

Many investigators have attempted to develop immune mediated methods of fertility control for humans, and much has been learned about successful and unsuccessful approaches (Talwar and Gaur, 1987; Alexander and Bialy, 1994). The goals of human contraceptive vaccines are substantially different and more difficult to achieve than an ideal dog or cat vaccine, and include requirements such as reversibility, no modification of reproductive behavior, and no detectable changes in the tissues of reproductive organs. Surprisingly little has been published about use of this obvious approach for control of pet overpopulation (Ladd et al., 1994). Although the specific objectives of immunocontraception are very different between humans and animals, the basic techniques are related.

For at least 30 years scientists have been developing contraceptive vaccines for use in humans, and substantial progress has been made, leading to phase II clinical trials currently in progress (Talwar and Gaur, 1987; Alexander and Bialy, 1994). One major impediment of immunocontraception is to trick the body into mounting an immune response against itself, in the form of hormones or structural components of eggs and sperm. This is much more problematic than designing vaccines for foreign antigens, such as infectious organisms. Other technical limitations of conventional protein based immunization include the need to highly purify compounds (e.g., hormones) which normally exist in very small quantities in the body, the difficulty in producing enough of these purified proteins to immunize an animal, let alone thousands or millions of animals, the problems of maintaining these temperature sensitive materials from manufacture to the point of use to assure their potency and effectiveness, and the obvious high cost of overcoming these problems.

Three methods are currently favored to achieve successful immunocontraception: induction of immunity against reproductive hormones, immunization against sperm antigens and immunity to the zona pellucida, a protein corona which surrounds the egg and facilitates fertilization by sperm. All three approaches have advantages and limitations, but used individually or in combination are likely to achieve the essential characteristics of a useful dog and cat contraceptive vaccine including: (a) prevention of fertility, (b) elimination of reproductive behavior, especially in females, i.e., prevention of the female going into “heat”, (c) long term effectiveness, preferably permanent (d) efficiencies exceeding 80% after initial immunization and approaching 100% after multiple vaccinations, (e) inexpensive to manufacture, (f) stable under field conditions, (g) easy to administer, and (h) free from serious non-reproductive health consequences.

Zona pellucida is an attractive target since antibodies to this protein interfere directly with fertilization and it is highly immunogenic across some species (eg porcine ZP3 is immunogenic for some other species) (Kaul et al., 1996; Mahi-Brown et al., 1985). Others have observed that ZP3 is reasonably antigenic, presumably because the protein is not expressed during infancy when tolerance is developed. Unfortunately, this vaccine has several limitations for use in dogs and cats. For example, it would affect only females and does not alter reproductive behavior. It has been shown that the native protein derived from swine is antigenic in cats, but does not interfere with fertility, presumably because the antigenic epitopes to which cats respond have no corresponding sites on the native feline zona pellucida (Gorman et al., 2002). Finally, to date only native proteins derived principally from pig ovary zona pellucida has been used as an immunogen, because recombinant protein derived from the native cDNA sequence lacks the post translational modification necessary for an antibody to interfere with native zona pellucida function. For these reasons, we believe that anti-zona pellucida vaccines are not appropriate for immunocontraception of dogs and cats.

GnRH is a decapeptide trophic hormone for both male and female reproduction. GnRH-specific immunization, therefore, can be used for both sexes (Fraser et al., 1974; Clark et al., 1978; Silversides et al., 1990; Jeffcoate et al., 1974; Hsu et al., 2000). Treatments that decrease GnRH would also likely suppress reproductive behavior. Because GnRH is a very small decapeptide and is recognized by the body as self, it presents a challenge to induce immunity. To circumvent this problem, GnRH can be linked to an antigenic carrier to enhance its immunological recognition and consequent immune response. For example, GnRH that is chemically conjugated to the antigenic carrier Tetanus toxoid or translationally conjugated to the antigenic carrier leukotoxin has been shown to induce anti-GnRH antibody responses. Moreover, antigenicity can be increased by altering the number of GnRH repeats. The most effective antigen includes 12-16 tandem repeats of GnRH, with some evidence that the longer the GnRH multimer, the greater the antibody response. Antigens with fewer copies require greater exposure (greater amount and more frequent booster immunization) to achieve sustained immunocontraception.

The present invention relates to developing optimized GnRH-containing immunogenic compositions for immunocontraception. The problem of enhancing the antigenicity of GnRH in these compositions has been solved, in part, by translationally conjugating a GnRH multimer to the immunogenic but non-toxic tetanus toxin C fragment, and by developing virus-vectored and bacterium-vectored vaccines. Moreover, the use of recombinant vectored vaccines that do not require a cold chain for storage represents a powerful solution to the problem of the effort and cost required at present to sterilize animals surgically, and the problem of the difficulty in applying present methods to large numbers of animals or animals that are free ranging.

In addition to providing improved methods for population control in free-ranging animals such as feral cats, the present invention is a solution to the problem of boar taint (i.e., the off-flavor and non-ideal organoleptic properties of meat from mature male animals). This method involves markedly less effort than surgical castration and, in contrast to surgical castration, can be used to control sexual physiology at more than one point during development The immunogenic compositions of the present invention may be more effective than prior art vaccines (e.g., U.S. Pat. No. 5,573,767), which do not utilize recombinant vectors as vaccine carriers.

The present invention can also be used to treat patients with GnRH-associated diseases such as GnRH-sensitive ovarian cancer and prostate cancer (Hsu et al., 2000).

### OBJECT AND SUMMARY OF THE INVENTION

The present invention relates to the use of recombinant vectors containing genetic sequences encoding multimers of GnRH alone or in combination with genetic sequences encoding bacterial toxins such as Clostridium tetani C toxin fragment, administered orally, topically, on the mucosa, or injected into body tissues by whatever means (e.g., by intradermal injection) to modify sexual behavior or fertility, or both, of vertebrates by inducing an immune response that alters normal physiologic sexual function.

The recombinant vectors are viruses such as adenovirus or bacteria such as Salmonella spp. or Escherichia spp. GnRH is normally produced in the hypothalamus and stimulates the pituitary to release lutenizing hormone and follicle stimulating hormone. The genetic sequence that encodes GnRH (a decapeptide) is short and by sequential linking of the DNA sequences encoding GnRH, multiple copies of the decapeptide can be encoded resulting in a “multimer”. Linking GnRH to an antigenic carrier (the bacterial toxin) enhances the immunological recognition. Efficacy of the recombinant vector(s) may be dependent on the route of administration. Therefore, the present invention relates to all routes of administration, given the fact that the vectors cited have been demonstrated to cause host cells to express the gene product(s) of the tetanus C toxin fragment (tetC):GnRH fusion protein or process the gene product(s) contained within the vector if administered by one, several, or all of the routes claimed. The present invention relates to modification of sexual behavior in treated males and females wherein libido is compromised. Vertebrates of either gender have decreased interest in or no desire to court, mate, and/or engage in sexual intercourse, spawn or fertilize. The present invention further relates to an immunological response by the host that alters the otherwise normal physiology associated with endocrine control of ovulation, maturation of spermatozoa, conception, and implantation.

Additionally, the linking of GnRH to an antigenic carrier (the bacterial toxin) may be accomplished by expression in one or more vectors, i.e., GnRH and the bacterial toxin may be expressed by the same vector, or they may be expressed by different vectors and administered together.

Additionally, the present invention relates to the use of antibodies to gene products(s) induced by the recombinants claimed above, passively, that cause the afore mentioned alterations in sexual physiology. These antibodies may be monoclonal and/or polyclonal and are produced by hybridomas, natural occurring or engineered cell lines, recombinant technology or by a treated host.

Furthermore, the present invention relates to the use of recombinant protein antigens consisting of multimers of GnRH in combination with bacterial toxins, adjuvants, oligonucleiotides containing CpG sequences to produce an immune response to alter sexual physiology and/or behavior. These antigens may be separate, mixed or encapsulated. Encapsulation, which, for example, can facilitate the use of the immunogenic compositions in bait drops for non-domesticated animals, may be micron or sub-micron in size using liposomes, water-lipid emulsions, or polymers. Changes in sexual behavior include prevention of animals going into “heat” and birds going into molt.

Additionally, the present invention relates to the use of recombinant protein antigens consisting of multimers of GnRH in combination with an antigen or antigens, or a vector or vectors expressing antigens consisting of multimers of GnRH in combination with an antigen or antigens, wherein the other antigens are antigens of pathogens of specific hosts, including those that are pathogens of cats and/or dogs.

Further, the present invention relates to the use of recombinant vectors or antigens produced by such vectors as described above, wherein they are used in any of the manners in which LH-RH or GnRH or analogs thereof are currently used, including as in herein cited documents.

The present invention still further relates to an antitumor immune response in the host that bears androgen-dependent prostate tumors, or GnRH-sensitive ovarian tumors, using recombinant vectors, antibodies or antigens administered as mentioned above.

Additionally, the present invention relates to a method for immunocastrating male domestic animals such as cattle, sheep, chickens and pigs to improve the organoleptic properties of the animals' meat (e.g., by minimizing boar taint).

### BRIEF DESCRIPTION OF THE FIGURES

FIG. 1. Potential reduction in pet overpopulation resulting from the application of an immunocontraceptive vaccine. (a) There are 124 million cats (58M) and dogs (66.2M) in the United States according to the American Pet Products Manufacturers Association's 1996 National Pet Owners Survey. (b) Exact birth statistics are not known, but the estimate of 25.5M dog and cat births per year used in this analysis suggests that there are only 4.1M breeders or 3.3% of the total dog and cat population. This estimate assumes an average litter size of 5 and only one litter per year, when in fact the average cat litter is 4-6 and can have as many as 3 litters per year (cumulative birth rate of 12-18 per year) and dogs average 6-10 pups per litter and can have 2 litters per year (cumulative birth rate of 12-20 per year). Therefore, assumptions used in this analysis may underestimate the magnitude of the unwanted dog and cat population. (c) These assumptions may underestimate the result if no additional intervention, such as immunocontraception is attempted, since they are based on a static number of breeders. In fact, a single breeding female and her progeny can hypothetically produce 67,000 dogs in six years and one breeding cat and her offspring can produce as many as 420,000 cats in seven years. (d) Male contraception could have a significant impact on the success of the proposed strategy and will be pursued in this project, but since inclusion of data related to male breeders is numerically small compared to females, these data were omitted for simplicity and clarity of presentation.

FIG. 2. Antibody response of 3 male cats to immunization with a GnRH-Carrier recombinant protein vaccine formulation. These data demonstrate that the antigen is immunogenic and that adjuvantation results in the highest and most prolonged anti-GnRH antibody response.

FIG. 3. Serum testosterone concentration in 3 male cats after immunization with the same GnRH protein vaccine used in FIG. 2. The three cats immunized with vaccine without adjuvantation had serum testosterone suppression for only 4 weeks and did not respond to the 44 week booster. All three cats responded after the booster at 5 weeks with nearly total suppression of testosterone. However, complete and prolonged suppression occurred after the 16 week booster and was sustained through 32 weeks. At that time two of three cats showed minimal testosterone levels, while one cat (7A68) remained completely suppressed. An additional booster given at 44 weeks produced complete suppression in all three cats which is sustained at the most recent sampling (62 weeks).

FIGS. 4 and 5. Six male dogs were immunized with two different GnRH-Carrier recombinant protein vaccines identified as 2717 or Z8N. Booster immunizations were given on weeks 3,8, and 21. Sustained suppression of testosterone was not observed until after the 4′ immunization, except one dog (Z8N #2) who had no detectable testosterone after the 2″° immunization. All dogs had total suppression of testosterone to undetectable levels after the 4″ immunization, indicating immunocastration.

FIG. 6. In a separate experiment one dog (391 B) immunized with 2717 antigen showed progressive reduction in plasma testosterone following the first booster given at two weeks and remained below 50 ng/dl from weeks 6 through 11 (last data point sampled).

FIG. 7. To determine the relationship between testosterone values and spermatogenesis, we evaluated testicular histology of dog 391B at week 11. The top panel shows the epididymis of the immunized dog compared with a normal control (Bottom panel). The epididymis of the normal testis is full of maturing and mature sperm, while that of the immunized dog are essentially devoid of sperm.

FIG. 8. Three cats were immunized by subcutaneous injection with AdCMV-tetC and antibody titer to TetC was assayed. Within two weeks two of three vaccinates has a titer of 1:400. By 4 weeks all three had titers of 1:400 or higher and by 5 weeks all three had titers of 1:6400.

FIG. 9. Three cats were immunized by intramuscular injection with AdCMV-tetC and antibody titer to TetC was assayed. Within 2 weeks 2 of 3 had titers of 1:1600 or 1:6400. At 5 weeks these same two cats had titers of 1:25,600. One cat failed to respond at all.

FIG. 10. Three cats were immunized by intranasal installation with AdCMV-tetC and antibody titer to TetC was assayed. Within two weeks one vaccinate had a titer of 1:6400. By 4 weeks all three had titers of 1:400 or higher and by 5 weeks all three had titers of 1:6400 to 1:25,600.

FIG. 11. Anti-tetC antibodies generated by needle free vectored vaccines at 3 and 7 weeks post immunization as determined by Elisa titer.

FIG. 12. Anti-tetC antibodies generated by needle free vectored vaccines as determined by Elisa titer. Vaccination methods included non-invasive vaccination onto the skin (NIVS) of an adenovirus recombinant encoding the tetanus toxin C fragment and intranasal inoculation (IN) of the same adenovirus recombinant. These were compared against intramuscular (IM) injection of a plasmid expression vector encoding the tetanus toxin C fragment.

FIG. 13. The antibody response to anti-GnRH protein antigen with CpG adjuvant reached contraceptive level by two months in mature female cats (n=6; control n=2).

FIG. 14. Anti-GnRH protein antigen with CpG adjuvant arrested estrus cycling in a postpubescent female cat approximately one month following vaccination.

FIGS. 15 and 16. Anti-GnRH protein antigen with CpG adjuvant prevented initiation of estrus cycling in a prepubescent female cat.

FIGS. 17 and 18. Anti-GnRH protein antigen with CpG adjuvant reduced serum testosterone to undetectable levels in male cats.

FIG. 19. Anti-GnRH protein antigen with CpG adjuvant prevented development of secondary sex characteristics in male cats immunized before puberty.

FIG. 20. Anti-GnRH protein antigen with CpG adjuvant induced anti-GnRH antibody titers sufficient to arrest testicular development in prepubescent male cats.

FIG. 21. Anti-GnRH protein antigen with CpG adjuvant induced body condition in vaccinates similar to spayed female cats.

FIG. 22. Anti-GnRH protein antigen with CpG adjuvant and involuted ovaries and uteri of vaccinates.

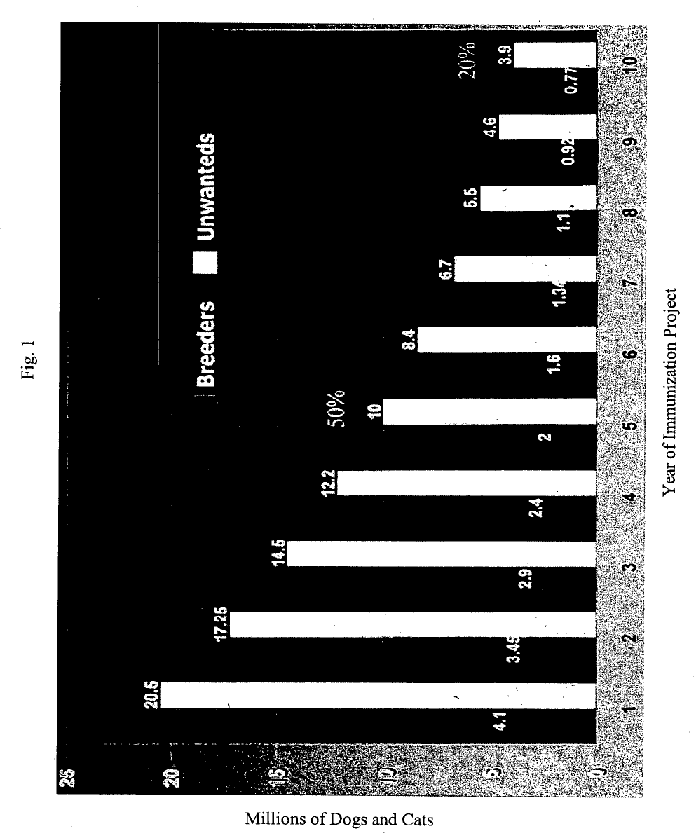

### DETAILED DESCRIPTION OF THE INVENTION

A completely novel approach must be devised which fulfills the ideal criteria outlined above. Tetanus toxoid (TT) has been used extensively in anti-GnRH vaccine development because TT is a member of the super antigen class and serves well as a “carrier” antigen (Chengalvala et al., 1999). Vaxin, Inc has developed a vaccine vectored by human adenovirus (Ad) which expresses the non-toxic tetanus toxin C fragment. AdCMV-tetC is described in U.S. Pat. No. 6,348,450 B1 issued Feb. 19, 2002. The original purpose of this product was immunization of humans against illness and death due to the toxin produced by the organism Clostridium tetani resulting from contaminated wounds. This vaccine has been successful in producing anti-tetC antibody in mice which is protective against Clostridium tetani challenge. The combined effectiveness of this AdCMV-tetC vaccine with the addition of a multimer of GnRH is believed to be exactly the novel approach which may provide the vigorous, sustained immune response required for an effective anti-GnRH immunocontraceptive vaccine. See also Makoffet al. (1989) for a description of TetC optimization for expression in E. coli, and Shi et al. (2001) for modification of synthetic TetC [Makoff, A. J., Oxer, M. D., Romanos, M. A., Fairweathre, N. F., and Ballantine, S. Expression of tetanus toxin fragment C in E. coli: high level expression by removing rare codons. Nucleic Acids Res. 17, 10191-10202 (1989); Shi, Z., Zeng, M., Yang, G., Siegel, F., Cain, L. J., Van Kampen, K. R., and Tang, D. C. Protection against tetanus by needle-free inoculation of adenovirus-vectored nasal and epicutaneous vaccines. J. Virol. 75, 11474-11482 (2001)].

We have a method to produce a GnRH multimer cassette which provides a technical advantage for rapid construction of multimers of any size. We use primers which produce restriction enzyme sites which allow cloning into different locations including a Kozak consensus translation start site. Thus, with these primers we have the ability to add GnRH multimers to the 5′ end of the coding sequence of any carrier molecule. With this method we have produced a GnRH with 14 repeats which is used in constructing the AdTetC-GnRH vaccine.

We have evaluated anti-GnRH vaccines in dogs and cats using assays of reproductive hormones, anti-GnRH antibodies, reproductive behavior, fertility and histological changes in reproductive organs. A radioimmunoassay is used to assay the immunological response of dogs treated with the GnRH vaccine.

One antigen was designed by inserting multiple copies of GnRH into proteins of E. coli at the precise locations known to be the primary immunogenic sites. This antigen did not achieve gonadal suppression for our target of 12 months. We believe that by using adenovirus, E. coli, poxvirus or Salmonella as vectors and TetC as the antigenic carrier we can achieve this goal.

As used herein, a vector is a tool that allows or facilitates the transfer of an entity from one environment to another. By way of example, some vectors used in recombinant DNA techniques allow entities, such as a segment of DNA (such as a heterologous DNA segment, such as a heterologous cDNA segment), to be transferred into a target cell. In an advantageous embodiment, the vector includes a viral vector, a bacterial vector, a protozoan vector, a DNA vector, or a recombinant thereof.

Reference is made to U.S. Pat. No. 5,990,091 issued Nov. 23, 1999, Einat et al. or Quark Biotech, Inc., WO 99/60164, published Nov. 25, 1999 from PCT/US99/11066, filed May 14, 1999, Fischer or Rhone Merieux, Inc., WO98/00166, published Jan. 8, 1998 from PCT/US97/11486, filed Jun. 30, 1997 (claiming priority from U.S. applications Ser. Nos. 08/675,556 and 08/675,566), van Ginkel et al., J. Immunol 159(2):685-93 (1997) (“Adenoviral gene delivery elicits distinct pulmonary-associated T helper cell responses to the vector and to its transgene”), and Osterhaus et al., Immunobiology 184(2-3):180-92 (1992) (“Vaccination against acute respiratory virus infections and measles in man”), for information concerning expressed gene products, antibodies and uses thereof, vectors for in vivo and in vitro expression of exogenous nucleic acid molecules, promoters for driving expression or for operatively linking to nucleic acid molecules to be expressed, method and documents for producing such vectors, compositions comprising such vectors or nucleic acid molecules or antibodies, dosages, and modes and/or routes of administration (including compositions for nasal administration), inter alia, which can be employed in the practice of this invention; and thus, U.S. Pat. No. 5,990,091 issued Nov. 23, 1999, Einat et al. or Quark Biotech, Inc., WO 99/60164, published Nov. 25, 1999 from PCT/US99/11066, filed May 14, 1999, Fischer or Rhone Merieux, Inc., WO98/00166, published Jan. 8, 1998 from PCT/US97/11486, filed Jun. 30, 1997 (claiming priority from U.S. applications Ser. Nos. 08/675,556 and 08/675,566), van Ginkel et al., J. Immunol 159(2):685-93 (1997) (“Adenoviral gene delivery elicits distinct pulmonary-associated T helper cell responses to the vector and to its transgene”), and Osterhaus et al., Immunobiology 184(2-3):180-92 (1992) (“Vaccination against acute respiratory virus infections and measles in man”) and all documents cited or referenced therein and all documents cited or referenced in documents cited in each of 5,990,091 issued Nov. 23, 1999, Einat et al. or Quark Biotech, Inc., WO 99/60164, published Nov. 25, 1999 from PCT/US99/11066, filed May 14, 1999, Fischer or Rhone Merieux, Inc., WO98/00166, published Jan. 8, 1998 from PCT/US97/11486, filed Jun. 30, 1997 (claiming priority from U.S. applications Ser. Nos. 08/675,556 and 08/675,566), van Ginkel et al., J. Immunol 159(2):685-93 (1997) (“Adenoviral gene delivery elicits distinct pulmonary-associated T helper cell responses to the vector and to its transgene”), and Osterhaus et al., Immunobiology 184(2-3):180-92 (1992) (“Vaccination against acute respiratory virus infections and measles in man”) are hereby incorporated herein by reference. Information in U.S. Pat. No. 5,990,091 issued Nov. 23, 1999, WO 99/60164, WO98/00166, van Ginkel et al., J. Immunol 159(2):685-93 (1997), and Osterhaus et al., Immunobiology 184(2-3):180-92 (1992) can be relied upon for the practice of this invention (e.g., expressed products, antibodies and uses thereof, vectors for in vivo and in vitro expression of exogenous nucleic acid molecules, exogenous nucleic acid molecules encoding epitopes of interest or antigens or therapeutics and the like, promoters, compositions comprising such vectors or nucleic acid molecules or expressed products or antibodies, dosages, inter alia). It is noted that immunological products and/or antibodies and/or expressed products obtained in accordance with this invention can be expressed in vitro and used in a manner in which such immunological and/or expressed products and/or antibodies are typically used, and that cells that express such immunological and/or expressed products and/or antibodies can be employed in in vitro and/or ex vivo applications, e.g., such uses and applications can include diagnostics, assays, ex vivo therapy (e.g., wherein cells that express the gene product and/or immunological response are expanded in vitro and reintroduced into the host or animal), etc., see U.S. Pat. No. 5,990,091, WO 99/60164 and WO 98/00166 and documents cited therein. Further, expressed antibodies or gene products that are isolated from herein methods, or that are isolated from cells expanded in vitro following herein administration methods, can be administered in compositions, akin to the administration of subunit epitopes or antigens or therapeutics or antibodies to induce immunity, stimulate a therapeutic response and/or stimulate passive immunity. The quantity to be administered will vary for the patient (host) and condition being treated and will vary from one or a few to a few hundred or thousand micrograms, e.g., 1 μg to 1 mg, from about 100 ng/kg of body weight to 100 mg/kg of body weight per day and preferably will be from 10 μg/kg to 10 mg/kg per day. A vector can be non-invasively administered to a patient or host in an amount to achieve the amounts stated for gene product (e.g., epitope, antigen, therapeutic, and/or antibody) compositions. Of course, the invention envisages dosages below and above those exemplified herein, and for any composition to be administered to an animal or human, including the components thereof, and for any particular method of administration, it is preferred to determine therefore toxicity, such as by determining the lethal dose (LD) and LD50 in a suitable animal model e.g., rodent such as mouse; and, the dosage of the composition(s), concentration of components therein and timing of administering the composition(s), which elicit a suitable response, such as by titrations of sera and analysis thereof, e.g., by ELISA and/or seroneutralization analysis. Such determinations do not require undue experimentation from the knowledge of the skilled artisan, this disclosure and the documents cited herein. And, the invention also comprehends sequential administration of inventive compositions or sequential performance of herein methods, e.g., periodic administration of inventive compositions such as in the course of therapy or treatment for a condition and/or booster administration of immunological compositions and/or in prime-boost regimens; and, the time and manner for sequential administrations can be ascertained without undue experimentation. Further, the invention comprehends compositions and methods for making and using vectors, including methods for producing gene products and/or immunological products and/or antibodies in vivo and/or in vitro and/or ex vivo (e.g., the latter two being, for instance, after isolation therefrom from cells from a host that has had a non-invasive administration according to the invention, e.g., after optional expansion of such cells), and uses for such gene and/or immunological products and/or antibodies, including in diagnostics, assays, therapies, treatments, and the like. Vector compositions are formulated by admixing the vector with a suitable carrier or diluent; and, gene product and/or immunological product and/or antibody compositions are likewise formulated by admixing the gene and/or immunological product and/or antibody with a suitable carrier or diluent; see, e.g., U.S. Pat. No. 5,990,091, WO 99/60164, WO 98/00166, documents cited therein, and other documents cited herein, and other teachings herein (for instance, with respect to carriers, diluents and the like).

Reference is made to patents pertaining to additional methods and compositions pertaining for contraception by vaccination, the texts of which are hereby incorporated herein by reference: EP0270056; EP0558631; EP0461177; EP0646015; U.S. Pat. Nos. 5,348,866, 5,989,550, 5,656,488, 5,637,300, 6,045,799, 6,027,727, 6,022,960, 4,676,981, 5,723,129, 5,684,145, and 5,573,767; WO 93/14786; WO 00/15253; WO 98/36073; and WO 93/14786. For example, U.S. Pat. No. 6,022,960 pertains to GnRH-leukotoxin chimeras and their use in vaccines, and U.S. Pat. No. 5,684,145 pertains to GnRH conjugates to bacterial fimbrial filaments and their use in vaccines.

In addition, various journal articles pertain to immunization and contraception, the texts of which are hereby incorporated herein by reference. Topics covered by these articles include: failure of female baboons (papio anubis) to conceive following immunization with recombinant non-human primate zona pellucida glycoprotein-B expressed in E. coli. Govind, C. K.; Gupta, S. K. Vaccine 18 (26) pp 2970-8 2000; antigen-specific systemic and reproductive tract antibodies in foxes immunized with Salmonella typhimurium expressing bacterial and sperm proteins. de Jersey J, et al. Reprod Fertil Dev 11 (4-5) pp 219-28 1999; infertility in female rabbits (Oryctolagus cuniculus) alloimmunized with the rabbit zona pellucida protein ZPB either as a purified recombinant protein or expressed by recombinant myxoma virus. Kerr, P. J. et al. Biol Reprod 61 (3) pp 606-13 1999; development of a recombinant ovalbumin-lutenizing hormone releasing hormone as a potential sterilization vaccine. Zhang, Y. et al. Vaccine 17 (17) pp 2185-91 1999; recombinant fertilization antigen-1 causes a contraceptive effect in actively immunized mice. Naz, R. K.; Zhu, X. Biol Reprod 59 (5) pp 1095-100 1998; Zonagen suspends further research on zona pellucida contraceptive vaccine. Business Wire, Sep. 27, 2001.

With respect to exogenous DNA for expression in a vector (e.g., encoding an epitiope of interest and/or an antigen and/or a therapeutic) and documents providing such exogenous DNA, as well as with respect to the expression of transcription and/or translation factors for enhancing expression of nucleic acid molecules, and as to terms such as “epitope of interest”, “therapeutic”, “immune response”, “immunological response”, “protective immune response”, “immunological composition”, “immunogenic composition”, and “vaccine composition”, inter alia, reference is made to U.S. Pat. No. 5,990,091 issued Nov. 23, 1999, and WO 98/00166 and WO 99/60164, and the documents cited therein and the documents of record in the prosecution of that patent and those PCT applications; all of which are incorporated herein by reference. Thus, U.S. Pat. No. 5,990,091 and WO 98/00166 and WO 99/60164 and documents cited therein and documents or record in the prosecution of that patent and those PCT applications, and other documents cited herein or otherwise incorporated herein by reference, can be consulted in the practice of this invention; and, all exogenous nucleic acid molecules, promoters, and vectors cited therein can be used in the practice of this invention. In this regard, mention is also made of U.S. Pat. Nos. 6,004,777, 5,997,878, 5,989,561, 5,976,552, 5,972,597, 5,858,368, 5,863,542, 5,833,975, 5,863,542, 5,843,456, 5,766,598, 5,766,597, 5,762,939, 5,756,102, 5,756,101, 5,494,807.

Embodiments of the invention that employ adenovirus recombinants, may include E1-defective, E3-defective, and/or E4-defective adenovirus vectors, or the “gutless” adenovirus vector in which all viral genes are deleted. The E1 mutation raises the safety margin of the vector because E1-defective adenovirus mutants are replication incompetent in non-permissive cells. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. The “gutless” adenovirus vector is the latest model in the adenovirus vector family. Its replication requires a helper virus and a special human 293 cell line expressing both E1a and Cre, a condition that does not exist in natural environment; the vector is deprived of all viral genes, thus the vector as a vaccine carrier is non-immunogenic and may be inoculated for multiple times for re-vaccination. The “gutless” adenovirus vector also contains 36 kb space for accommodating transgenes, thus allowing co-delivery of a large number of antigen genes into cells. Specific sequence motifs such as the RGD motif may be inserted into the H-I loop of an adenovirus vector to enhance its infectivity. An adenovirus recombinant is constructed by cloning specific transgenes or fragments of transgenes into any of the adenovirus vectors such as those described above. The adenovirus recombinant is used to transduce epidermal cells of a vertebrate in a non-invasive mode for use as an immunizing agent.

The vaccines of the present invention can be administered to an animal either alone or as part of an immunological composition. For example, the vaccination can be combined with vaccines for other maladies which afflict domestic or other animals.

As to “immunogenic composition”, “immunological composition” and “vaccine”, an immunological composition containing the vector (or an expression product thereof) elicits an immunological response, local or systemic. The response can, but need not be protective. An immunogenic composition containing the inventive recombinant or vector (or an expression product thereof) likewise elicits a local or systemic immunological response which can, but need not be, protective. A vaccine composition elicits a local or systemic protective response. Accordingly, the terms “immunological composition” and “immunogenic composition” include a “vaccine composition” (as the two former terms can be protective compositions). The invention comprehends immunological, immunogenic or vaccine compositions.

With respect to dosages, routes of administration, formulations, adjuvants, and uses for recombinant viruses and expression products therefrom, compositions of the invention may be used for parenteral or mucosal administration, preferably by intradermal, subcutaneous or intramuscular routes. When mucosal administration is used, it is possible to use oral, ocular or nasal routes.

The inventive recombinant vector or immunological or vaccine compositions or therapeutic compositions, can be prepared in accordance with standard techniques well known to those skilled in the pharmaceutical or veterinary art. Such compositions can be administered in dosages and by techniques well known to those skilled in the veterinary arts taking into consideration such factors as the age, sex, weight, and the route of administration. The compositions can be administered alone, or can be co-administered or sequentially administered with compositions, e.g., with “other” immunological composition, or attenuated, inactivated, recombinant vaccine or therapeutic compositions thereby providing multivalent or “cocktail” or combination compositions of the invention and methods employing them. Again, the ingredients and manner (sequential or co-administration) of administration, as well as dosages can be determined taking into consideration such factors as the age, sex, weight, and, the route of administration. In this regard, reference is made to U.S. Pat. No. 5,843,456, incorporated herein by reference, and directed to rabies compositions and combination compositions and uses thereof such as bait drops; see also other documents cited herein and documents cited or referenced in herein cited documents, including U.S. Pat. No. 6,217,883.

Examples of compositions of the invention include liquid preparations for mucosal administration, e.g., oral, nasal, ocular, etc., administration such as suspensions and, preparations for parenteral, subcutaneous, intradermal, intramuscular (e.g., injectable administration) such as sterile suspensions or emulsions. In such compositions the recombinant poxvirus or immunogens may be in admixture with a suitable carrier, diluent, or excipient such as sterile water, physiological saline, or the like. The compositions can also be lyophilized or frozen. The compositions can contain auxiliary substances such as wetting or emulsifying agents, pH buffering agents, adjuvants, preservatives, and the like, depending upon the route of administration and the preparation desired. The compositions can contain at least one adjuvant compound

Preferably, a solution of adjuvant according to the invention, especially of carbomer, is prepared in distilled water, preferably in the presence of sodium chloride, the solution obtained being at acidic pH. This stock solution is diluted by adding it to the desired quantity (for obtaining the desired final concentration), or a substantial part thereof, of water charged with NaCl, preferably physiological saline (NaCL 9 g/l) all at once in several portions with concomitant or subsequent neutralization (pH 7.3 to 7.4), preferably with NaOH. This solution at physiological pH will be used as it is for mixing with the vaccine, which may be especially stored in freeze-dried, liquid or frozen form.

The compositions of the invention can also be formulated as oil in water or as water in oil in water emulsions, e.g. as in V. Ganne et al. Vaccine 1994, 12, 1190-1196.

Standard texts, such as “REMINGTON'S PHARMACEUTICAL SCIENCE”, 17th edition, 1985, incorporated herein by reference, may be consulted to prepare suitable preparations, without undue experimentation.

Compositions in forms for various administration routes are envisioned by the invention. And again, the effective dosage and route of administration are determined by known factors, such as age, sex, weight, and other screening procedures which are known and do not require undue experimentation. Dosages of each active agent can be as in herein cited documents (or documents referenced or cited in herein cited documents) and/or can range from one or a few to a few hundred or thousand micrograms, e.g., 1 μg to 1 mg, for a subunit immunogenic, immunological or vaccine composition.

Recombinant vectors can be administered in a suitable amount to obtain in vivo expression corresponding to the dosages described herein and/or in herein cited documents.

Suitable dosages can also be based upon the examples below.

### EXAMPLES

The invention will now be further described by the following non-limiting examples.

### Example 1

### Feline and Canine Responses to Anti-GnRH Antigen

We have evaluated anti-GnRH vaccines in dogs and cats using assays of reproductive hormones, anti-GnRH antibodies, reproductive behavior, fertility and histological changes in reproductive organs. A radioimmunoassay is used to assay the immunological response of dogs treated with the GnRH vaccine. GnRH labeled with radioiodine (I-125) is reacted with dilutions of sera from immunized subjects. Bovine serum albumin blocks nonspecific antigen-antibody binding. Antibody titer is defined as percentage of total radio labeled isotope bound in antibody containing sera that is precipitated with ethanol. Hormone concentrations are assayed in plasma or feces for estrone, estradiol, progesterone, and testosterone by radioimmunoassays. Male and female dogs immunized with constructs are examined for production of anti-GnRH antibodies. Female dogs are examined by vaginal cytology and plasma or fecal concentrations of estrogens and progesterones. Essentially the same procedures are used to test efficacy and safety in cats, except that vaginal cytology is not used because cats are induced ovulators. We have developed a method for estrogen and progesterone assays in feces. This assay provides endocrine measures that eliminate day to day fluctuations. Immunized males are examined for breeding soundness by microscopic examination of semen and testicular histology. We have developed standard methods which are optimal for reproductive success in these species. All dogs and cats are housed in runs, preferably in small groups. Illumination is carefully controlled for spectral wavelength (natural daylight spectrum bulbs), intensity and light dark cycle (14:10 L:D). Female vaccinates are housed with fertile males and observed for reproductive behavior and fertility. After immunization and detection of serum antibodies, dogs are in breeding groups of two immunized and one control female with an untreated fertile male. Cats are housed with all four test females, two controls and one tom. Female dogs are sampled weekly for vaginal cytology which will indicate the stage and impending changes in estrus cycle. Female dogs and cats are observed daily for indications of estrum characteristic of the species. Ovaries and testes are examined histologically for immune-system-dependent lesions. Unilateral ovarian and testicular gonadectomies are performed following observation of reproductive disruption by the assessments described above. The gonads are sectioned and examined for lesions. If lesions are found, they are characterized for type of cellular infiltrate and loss of primordial gamete and endocrine cells.

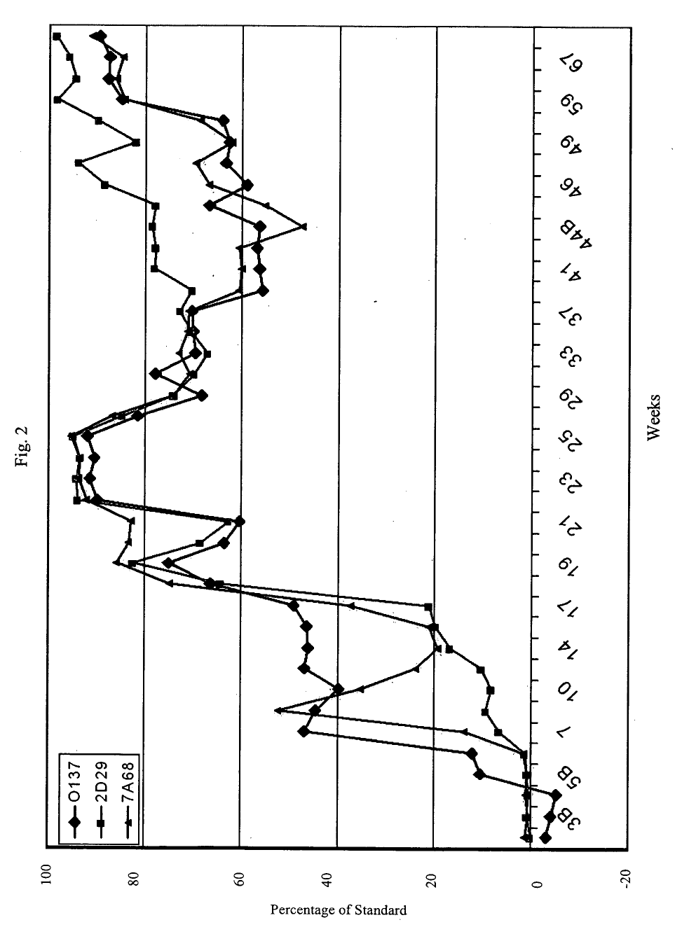

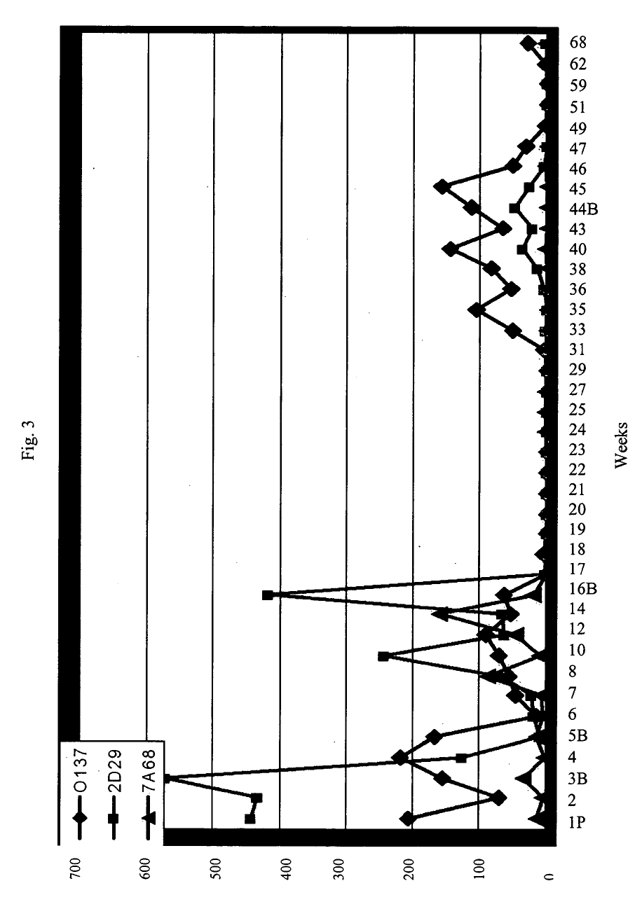

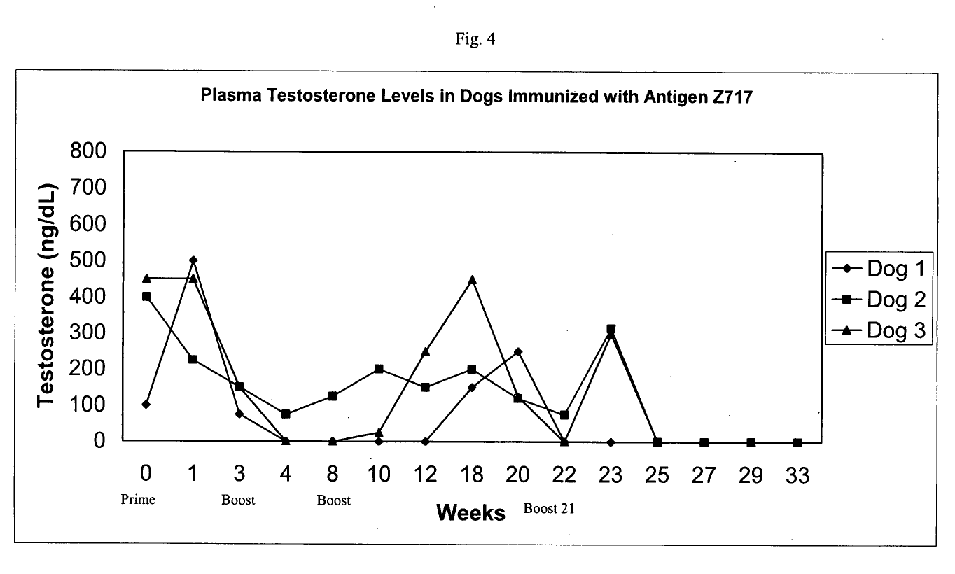

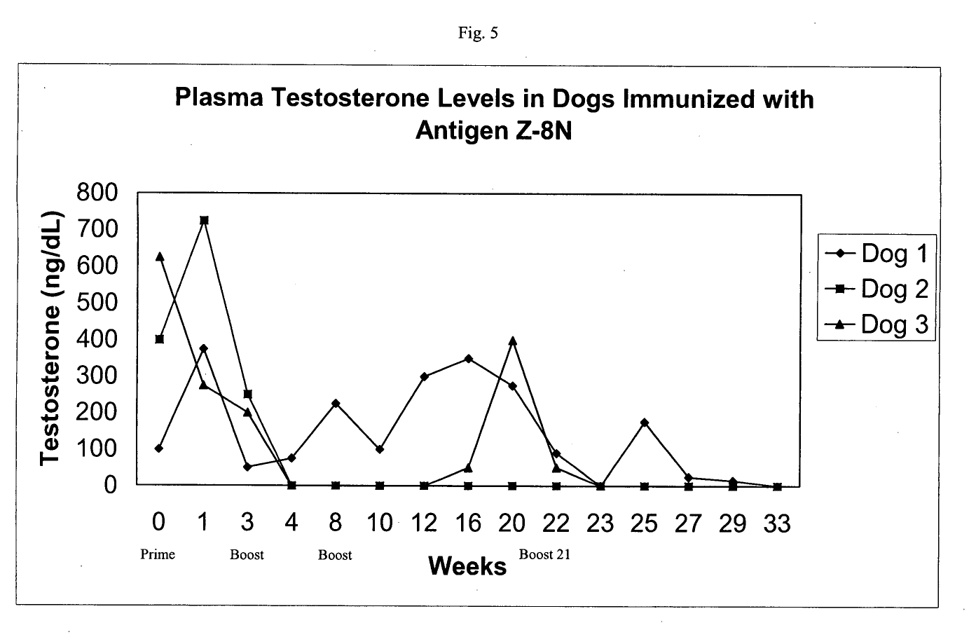

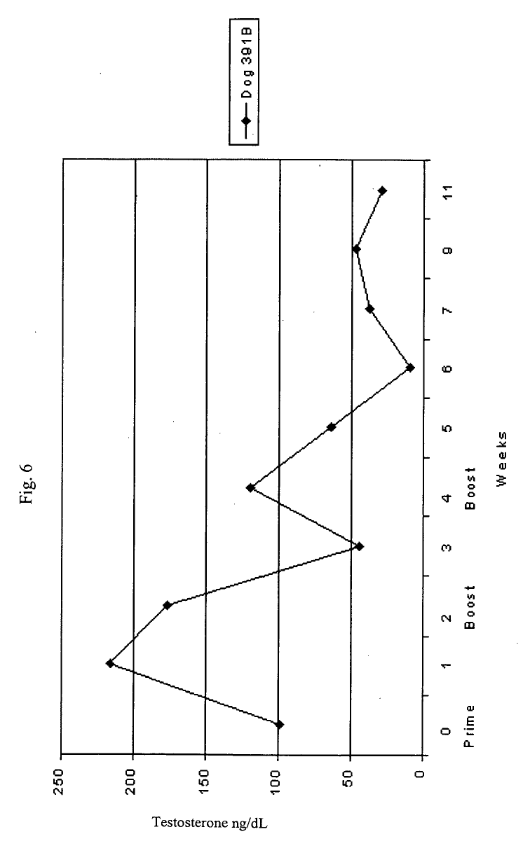

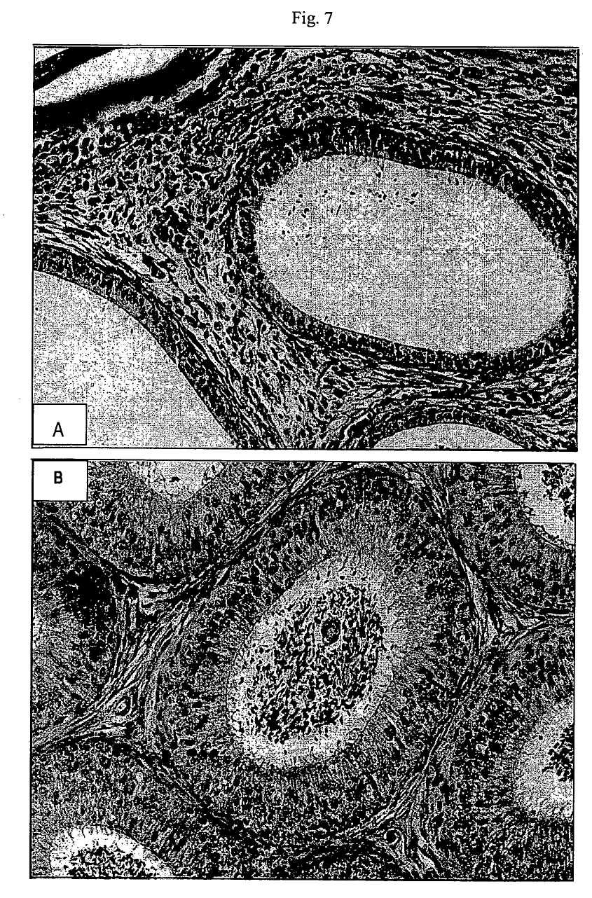

One antigen was designed by inserting multiple copies of GnRH into proteins of E. coli at the precise locations known to be the primary immunogenic sites. We have tested these antigens extensively for antigenicity, dose response, formulation and duration of gonadal suppression, with and without CpG molecular adjuvantation. We now have 18 months of data from male cats immunized with this antigen (FIGS. 2 and 3). From this immunization trial we learned that the CpG molecular immunostimulatory adjuvant is essential to achieve contraceptive antibody titers and sustained gonadal suppression. Cats immunized with the antigen plus CpG achieved titers of greater than 95% of the standard anti-GnRH antibody titer and maintained that antibody level for more that 4 months. When these cats were given a booster vaccination, they displayed a typical immune memory (anamnestic) response resulting in elevation of antibody titer. When these cats' antibody titers remained above 90% of standard, there was no detectable serum testosterone and their testicular diameter was 50% of normal. These findings demonstrate that these cats are immunocastrated. Similar results were obtained for dogs immunized with the same antigen (FIGS. 4 and 5). One of these dogs developed spermiogenesis after immunization (FIGS. 6 and 7). This antigen, which was neither translationally conjugated to TetC nor administered with a recombinant vector, did not achieve gonadal suppression for our target of 12 months. The use of Ad as a vector and TetC as the antigenic carrier may provide gonadal suppression for at least 12 months.

### Example 2

### Feline and Canine Response to TetC Recombinant Vector

An adenovirus vectored vaccine which expresses the non-toxic tetanus toxin C fragment has been evaluated. Our first immunization trial with the Vaxin AdCMV-tetC vaccine was to confirm that it induces a vigorous immune response in cats and dogs. An additional objective was to determine the optimal route of administration. The early results of our immunization trial with the Vaxin AdCMV-tetC vaccine involved three groups of three cats each that were immunized with AdCMV-tetC by one of three routes: intranasal (IN), intramuscular (M) or subcutaneous (SQ). A non-immunized control cat in each group served as sentinel for accidental transmission of the viral vector.

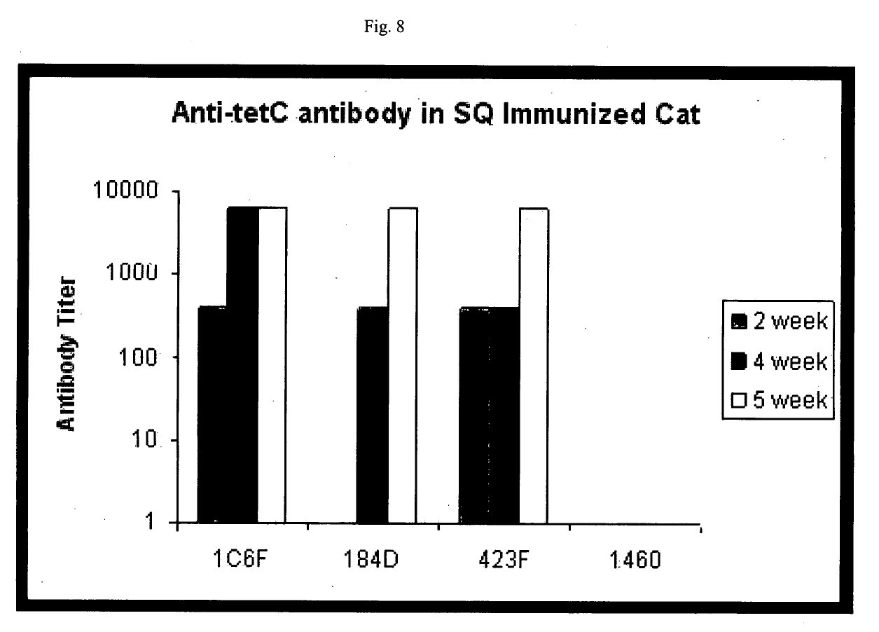

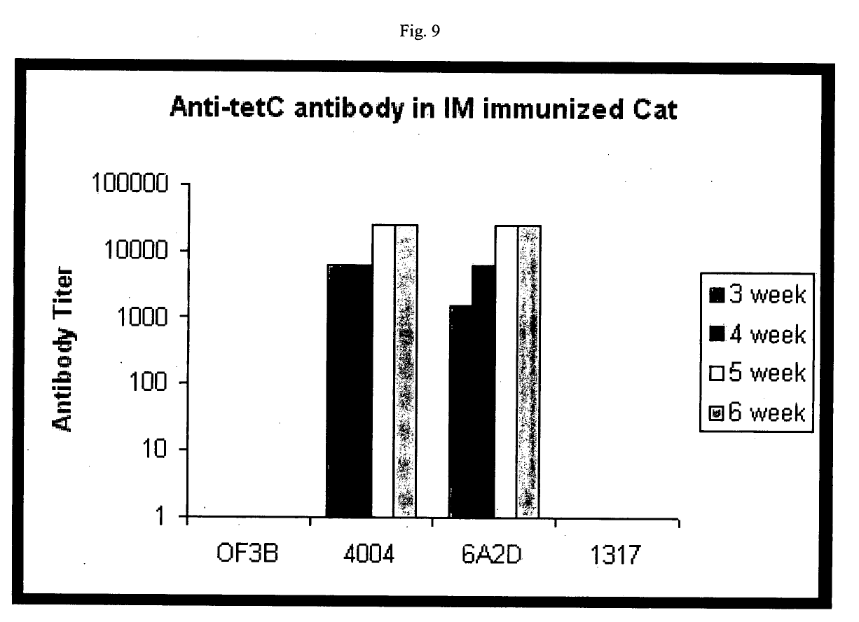

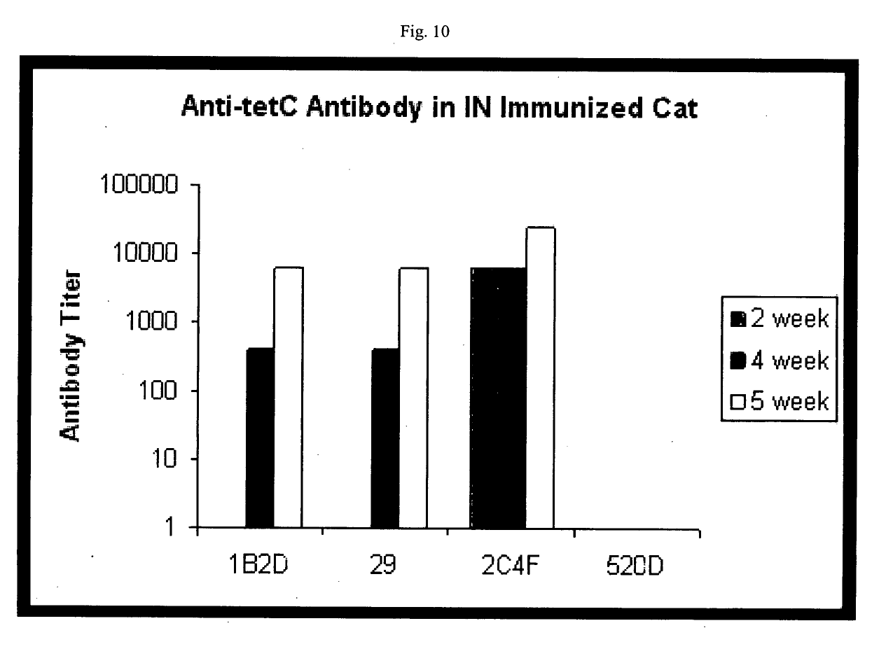

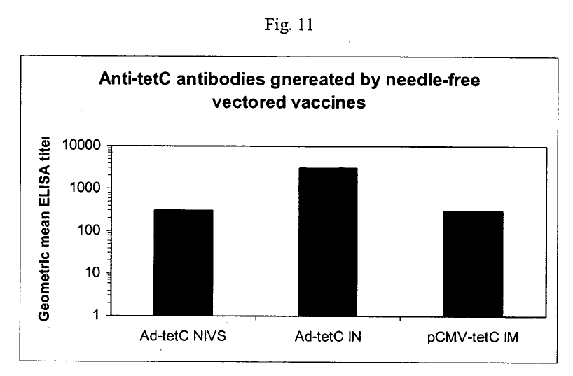

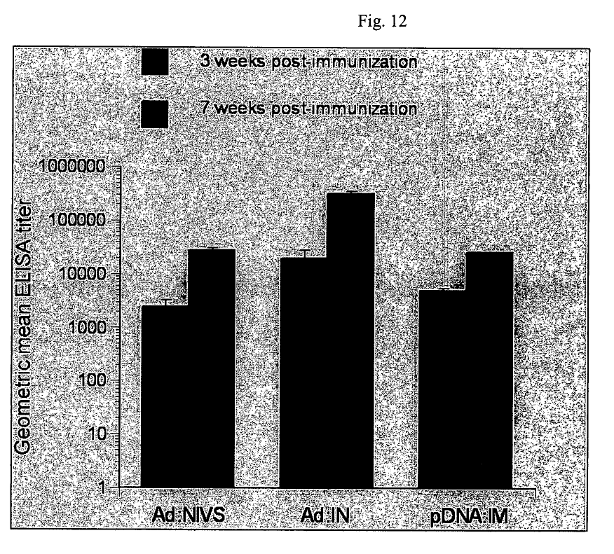

Cats in all three immunization groups responded vigorously and rapidly with high anti-tetanus antibody titers (see FIGS. 8-12). The three cats in the intramuscular immunization group developed high titers (1:6,400) within 3-4 weeks after a single primary vaccination; after a single booster administered at 4 weeks, the titers increased to an extremely high titer of 1:25,600 (FIG. 9). The 6 cats in the subcutaneous and intranasal groups all responded similarly after the booster to generate titers of 1:6,400 (5 of 6 cats) or 1:25,600 (1 of 6 cats) (FIGS. 8 and 10). None of the control cats, which were housed with the vaccinates, developed any detectable titer. This vectored product, therefore, is safe for the operator since there was no apparent accidental exposure of the control cats sufficient to induce antibody response.

Two groups of three dogs each were immunized with the AdCMV-tetC vector by one of two routes of administration, either 3IM or 3SQ, and two dogs served as unimmunized controls. The controls did not develop anti-TetC antibodies. All 6 immunized dogs developed antibody titers to the primary immunization. The mean titer was 1:250 for IM and SQ immunization, similar to the primary response in the corresponding cat study. After boosting at 8 weeks postimmunization, the titer for the three IM dogs was 1:1,250, and the titer for the three SQ dogs was 1:6,250.

The remarkable response to the primary immunizing dose, and robust response to the booster dose pointed towards the possibility of using an AdTetC-GnRH vaccine in a single dose immunization schedule.

### Example 3

### Plasmid Construction

Plasmid pGnRH-14 consists of 13.5 GnRH repeats inserted into the NcoI site of pTrueBlue-PvuII plasmid (FIG. 23). The GnRH repeats was excised with the NcoI restriction enzyme followed by in-frame insertion into the Ncol site of pCMV-tetC encoding the tetanus toxin C-fragment (tetC) (described in Shi et al., 2001) to create a GnRH:tetC fusion sequence driven by the cytomegalovirus (CMV) early promoter (pCMV-GnRH:tetC). For pCMV-tetC, see WO 00/66179 and U.S. Pat. No. 6,348,450, which are herein incorporated by reference.

GnRH Nucleotide/Peptide Sequences:

5′-GAA CAT TGG TCA TAT GGA CTA CGG CCG GGA-3′
E H W S Y G L R P G

### Example 4

### Construction of GnRH:tetC Recombinant Vectors

The GnRH:tetC fusion fragment was excised as a BamHI fragment from pCMV-GnRH:tetC and subsequently inserted into the BamHI site of pAdApt (Crucell) in the correct orientation (pAdApt-GnRH:tetC). For pAdApt, see U.S. Pat. Nos. 6,492,169, 6,447,768, and 6,340,595, which are herein incorporated by reference. A replication competent adenovirus (RCA)-free adenovirus vector encoding the GnRH:tetC fusion protein driven by the CMV promoter (AdCMV-GnRH:tetC) was constructed by co-transfecting pAdApt-GnRH:tetC with pJM17 into PER.C6 cells (Fallaux et al., 1998). Plaques were purified twice. AdCMV-GnRH:tetC was propagated and purified as described in Shi et al., 2001.

The GnRH:tetC fusion fragment was also excised as a BamHI fragment from pCMV-GnRH:tetC and subsequently inserted into the BamHI site of pnirB (constructed at Vaxin) in the correct orientation to create pnirB-GnRH:tetC for expression in a bacterial vector. An E. coli strain harboring pnirB-GnRH:tetC was grown in L broth containing 50 μg/ml kanamycin.

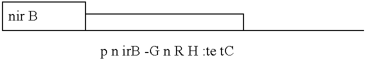

### Example 5

### Plasmid Construction

Plasmid pGnRH-14 contains 13.5 GnRH tandem repeats flanked by the NcoI site of pTrueBlue-PvuII. In pGnRH-14, a start methionine codon was situated directly upstream of the first EHWSYGLRPG GnRH repeat. The GnRH repeats were not interrupted by linker sequences, and the fourteenth repeat was truncated after EHWSYG.

The GnRH multimer sequence of pGnRH-14 was excised and introduced alone and in combination with TetC downstream of the pnirb bacterial promoter in a plasmid context and downstream of the cytomegalovirus immediate-early promoter in the adenovirus context.

The GnRH multimer replication-defective adenoviral recombinant vector was engineered by introducing an EcoRI-BamHI fragment containing the GnRH multimer sequence from plasmid ptrueblue-GnRH-14 into the EcoRI-BamHI site of pAdApt to produce pAdApt-GnRH (FIG. 24). The GnRH multimer nonpathogenic E. coli and Salmonella recombinant vectors were engineered by introducing a ClaI-BamHI fragment containing the GnRH multimer sequence from plasmid ptrueblue-GnRH-14 into the ClaI-BamHI site of pNirB to produce pNirB-GnRH, follwed by transformation of bacterial cells (FIG. 25).

The replication-defective adenoviral recombinant vector containing TetC translationally conjugated to the 3′ end of the GnRH multimer was engineered by introducing an NcoI fragment containing the GnRH multimer sequence from plasmid ptrueblue-GnRH-14 into the NcoI site of pBluscript-tetC to produce pbluscript-GnRH-tetC; the BamHI fragment of pbluscript-GnRH-tetC containing the GnRH-TetC fusion was then introduced into the BamHI site of pAdApt to produce pAdApt-GnRH-tetC (FIG. 26). The nonpathogenic E. coli and Salmonella recombinant vectors containing TetC translationally conjugated to the 3′ end of the GnRH multimer were engineered by introducing a BamHI fragment containing the GnRH-TetC fusion from plasmid ptrueblue-GnRH-tetC into the BamHI site of pNirB to produce pNirB-GnRH-tetC, followed by transformation of bacterial cells (FIG. 27).

### Example 6

### Feline Response to Anti-GnRH Protein Antigen and CpG Adjuvant

The effects of anti-GnRH protein antigen and CpG adjuvant on prepubescent cats of both genders and postpubescent female cats were tested in a one-year vaccine trial.

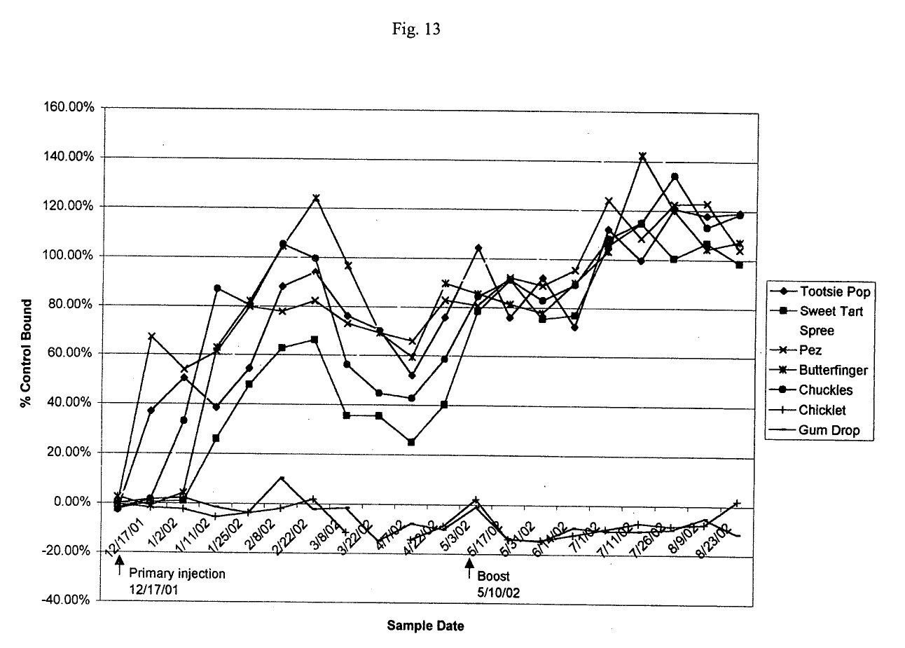

The antigen was a purified recombinant GnRH-leukotoxin chimera wherein P. haemolytica leukotoxin [Lo, R. Y., Shewen, P. E., Strathdee, C. A., and Greer, C. N. Cloning and expression of the leukotoxin gene of Pasteurella haemolytica A1 in Escherichia coli K-12. Infect. Immun. 50, 667-671 (1985)] was translationally conjugated to 8-copy multimers of GnRH at its N- and C-termini. The antigen was mixed with a stable oil-in-water emulsion. CpG optimized for cats [Wemette, C. M, Smith, B. F., Barksdale, Z. L., Hecker, R., and Baker, H. J. CpG oligodeoxynucleotides stimulate canine and feline immune cell proliferation. Vet. Immunol. Immunopathol. 84, 223-236 (2002)] was added to the antigen as an adjuvant. The formulated vaccine was administered subcutaneously in a volume of 0.25 ml containing 100 micrograms Ag and 100 micrograms CpG. Injection-site reactions of mild to moderate (0.5 cm) swelling were detected within 24 hours, and completely resolved within two weeks. Booster immunizations were administered as shown in FIGS. 13, 14, and 16.

The antibody response to anti-GnRH protein antigen with CpG adjuvant reached contraceptive level by two months (FIG. 13).

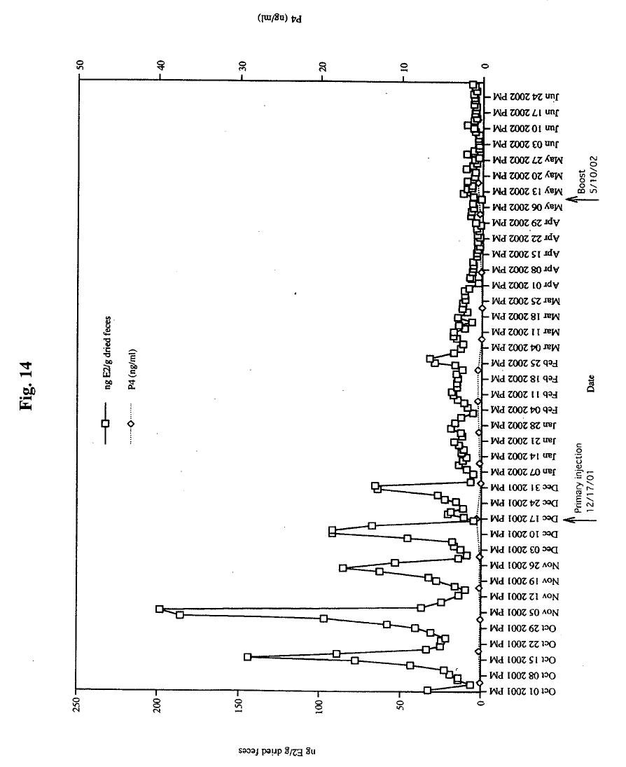

Anti-GnRH protein antigen with CpG adjuvant arrested estrus cycling in a postpubescent female (FIG. 14).

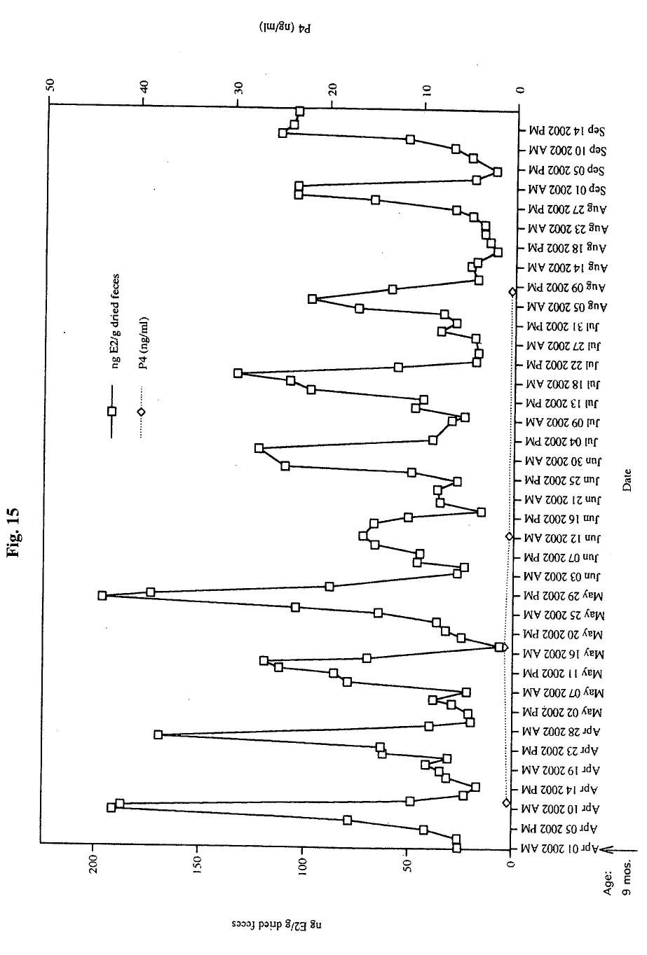

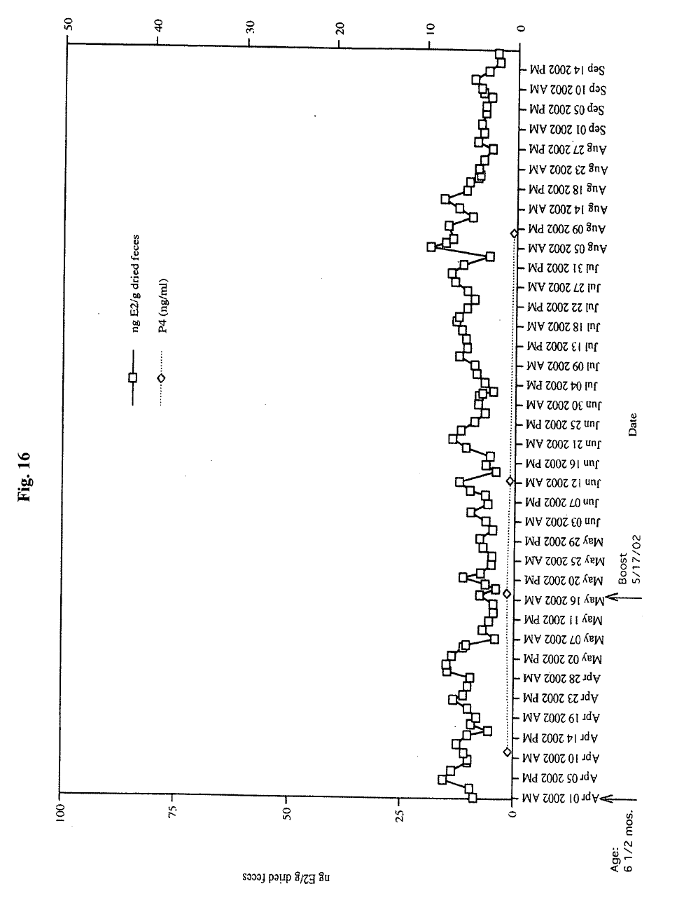

Anti-GnRH protein antigen with CpG adjuvant prevented initiation of estrus cycling in a prepubescent female (FIGS. 15 and 16).

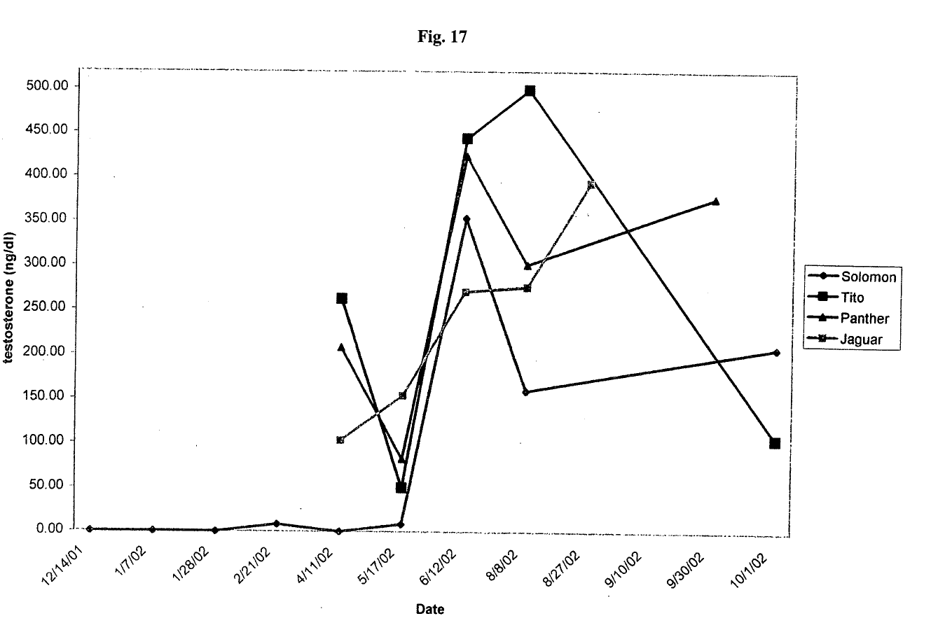

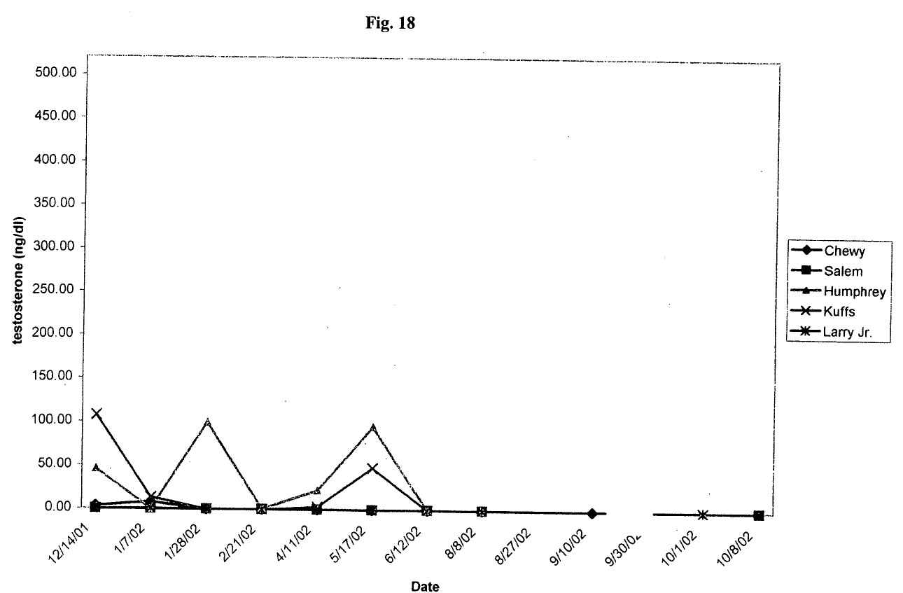

Anti-GnRH protein antigen with CpG adjuvant reduced serum testosterone to undetectable levels in males (FIGS. 17 and 18).

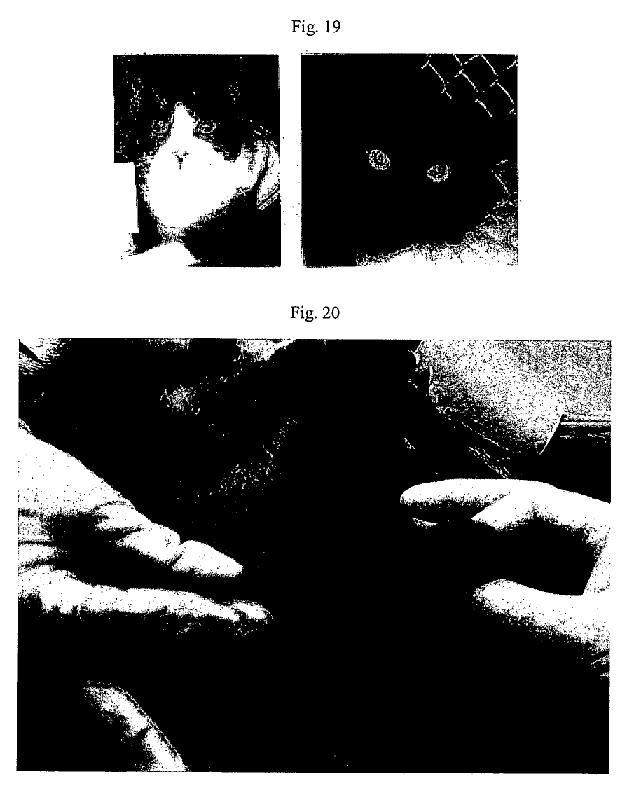

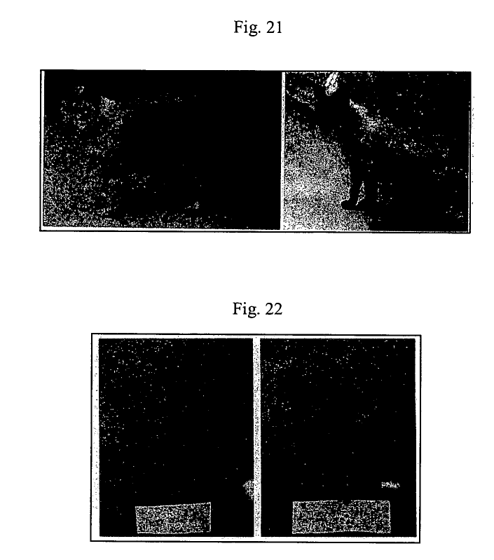

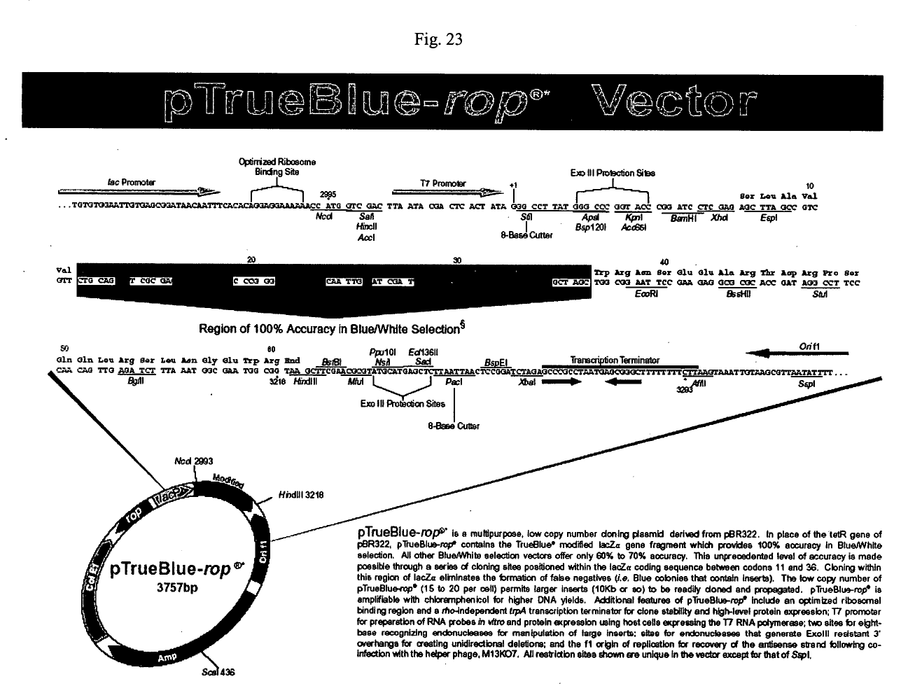

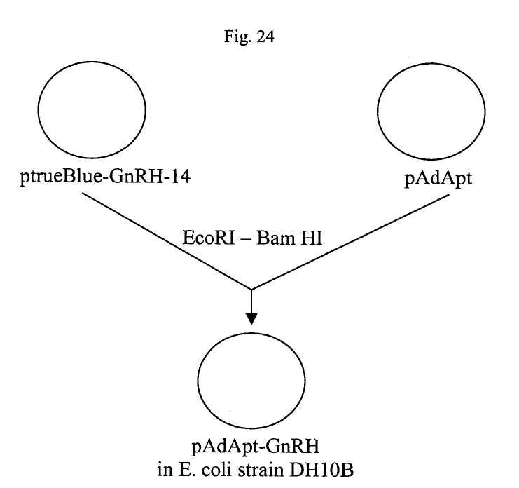

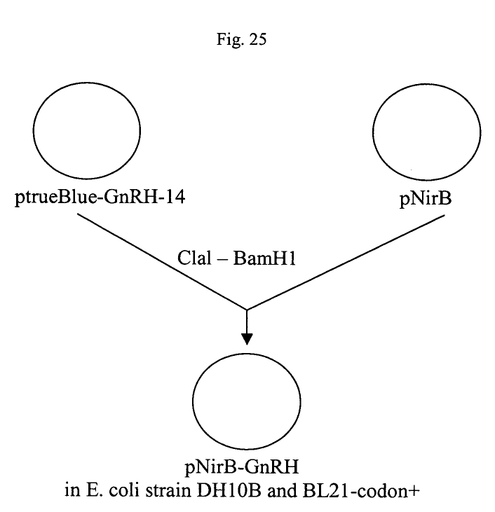

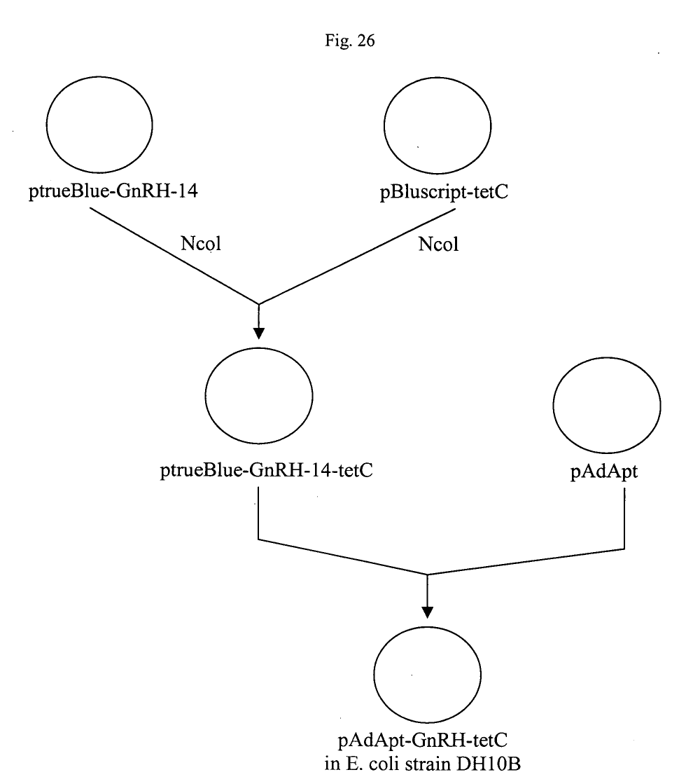

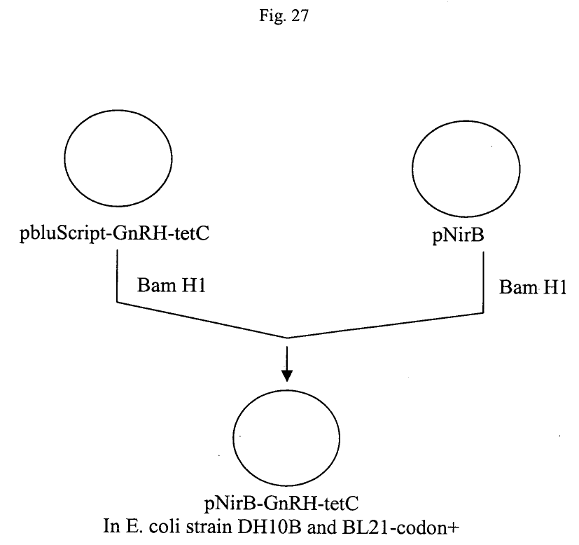

Anti-GnRH protein antigen with CpG adjuvant induced anti-GnRH antibody titers sufficient to prevented development of secondary sex characteristics in males immunized before puberty (FIG. 19), arrest testicular development in prepubescent males (FIG. 20), induced body condition in vaccinates similar to spayed females (FIG. 21), and involuted ovaries and uteri (FIG. 22).

### Example 7

### Mouse Serum Response to E. coli-Vectored GnRH Vaccines

Mice were immunized by topical application (5×10⁹ cfu per animal), intranasal instillation (1×10⁹ cfu per animal), or intramuscular injection (5×10⁹ cfu per animal) of an E. coli vector expressing GnRH or GnRH:tetC fusion driven by the nirB promoter. Animals were boosted once at an interval of 4 weeks. Sera were collected before immunization, 4 weeks after primary immunization before boost application, and 4 weeks postboost for analysis. Primary immunized sera following intranasal administration (n=4) contained similar levels of anti-GnRH antibodies relative to control mice (n=5), approximately 4% over control baseline; in contrast, boosted immunized sera (n=5) was approximately 37% over control baseline. Boosted immunized sera following topical administration (n=5) was approximately 20% over control baseline. Primary immunized sera following intramuscular administration (n=5) was approximately 93% over control baseline, and boosted immunized sera (n=4) was approximately 200% over baseline control.

In a second experiment, mice were immunized with a GnRH or GnRH-TetC bacterial recombinant vector, and binding of radiolabeled GnRH by their sera was determined. Intranasal administration of GnRH bacterial recombinant vector initially increased GnRH binding in 0 of 5 mice; boosted sera from 1 of these 5 mice exhibited increased GnRH binding (2.7% increase in percentage bound). Intranasal administration of GnRH-TetC recombinant vector initially increased GnRH binding in 1 of 5 mice (16% increase); boosted sera from 2 of these 5 mice exhibited increased GnRH binding (average 1.1% increase).

Intramuscular administration of GnRH bacterial recombinant vector initially increased GnRH binding in 0 of 5 mice; boosted sera from 0 of these 5 mice exhibited increased GnRH binding. Intramuscular administration of GnRH-TetC bacterial recombinant vector initially increased binding in 2 of 4 mice (average 2.5% increase); boosted sera from 3 of these 4 mice exhibited increased GnRH binding (average 7.8% increase).

Topical administration of GnRH bacterial recombinant vector initially increased GnRH binding in 0 of 5 mice; boosted sera from 1 of these 5 mice exhibited increased GnRH binding (3.3% increase). Topical administration of GnRH-TetC bacterial recombinant vector initially increased binding in 5 of 5 mice (average 8.9% increase); boosted sera from 1 of these 5 mice exhibited increased GnRH binding (1.0% increase).

### Example 8

### Canine Response to Anti-GnRH Protein Antigen

Three male Beagles were immunized with 400 ug of KLH:GnRH antigen administered intramuscularly. Responses were determined weekly following immunization (Table 1). At seven weeks after immunization, two of the three Beagles displayed 40% reduction in testicular size, no detectable testosterone, and no sperm production. The third Beagle displayed only 1% of its pre-immunization sperm count with only 10% of the remaining sperm having motility. Libido was not effected. It can be concluded that infertility was obtained by seven weeks after immunization. The effects of GnRH antigen on dogs, therefore, is similarly robust to its effects on cats. of its pre-immunization sperm count with only 10% of the remaining sperm having motility. Libido was not effected. It can be concluded that infertility was obtained by seven weeks after immunization. The effects of GnRH antigen on dogs, therefore, is similarly robust to its effects on cats.

TABLE 1
Response of three male Beagles 7 weeks after immunization with KLH: GnRH
antigen administered intramuscularly

| | | | | | Semen | | | | |
|---|---|---|---|---|---|---|---|---|---|
| | | | Testicle | | | | | | Normal |
| | | Testosterone | Size (cm) | | Sample | Volume | Concentration | Motility | morphology |
| Beagle | Date | (ng/dl) | Left | Right | obtained? | (ml) | (per ml) | (%) | (%) |
| Flash 0D2D | Jan. 16, 2003 | 94.07 | 2.5 | 2.50 | Yes | 3 | 150,000,000 | 75 | 83 |
| DOB: | Jan. 23, 2003 | 29.66 | 2.55 | 2.55 | N/A* | | | | |
| Jul. 25, 1998 | Jan. 30, 2003 | 123.99 | 2.50 | 2.50 | N/A | | | | |
| Dose: | Feb. 6, 2003 | 202.86 | 2.30 | 2.30 | N/A | | | | |
| 400 μg/0.5 ml | Feb. 13, 2003 | 40.21 | 2.15 | 2.15 | N/A | | | | |
| on 1/1703 | Feb. 20, 2003 | 154.51 | 2.20 | 2.20 | Yes | 2 | 2,050,000 | 50 | 68 |
| | Mar. 6, 2003 | 195.96 | 2.30 | 2.30 | Yes | 4.75 | 10,800,000 | 80 | 72 |
| | Mar. 21, 2003 | 123.25 | 2.10 | 2.30 | Yes | 4 | 2,100,000 | 10 | 82 |
| Teddy 5B54 | Jan. 16, 2003 | 88.88 | 2.30 | 2.30 | Yes | 2 | 144,000,000 | 88 | 94 |
| DOB: | Jan. 23, 2003 | 44.80 | 2.55 | 2.55 | N/A | | | | |
| Nov. 8, 2000 | Jan. 30, 2003 | 20.07 | 2.35 | 2.35 | N/A | | | | |
| Dose: | Feb. 6, 2003 | 13.45 | 2.35 | 2.35 | N/A | | | | |
| 400 μg/0.5 ml | Feb. 13, 2003 | 0.00 | 2.00 | 2.00 | N/A | | | | |
| on 1/1703 | Feb. 20, 2003 | 0.00 | 1.90 | 1.80 | No | N.D.* | | | |
| | Mar. 6, 2003 | 0.00 | 1.60 | 1.60 | No | N.D. | | | |
| | Mar. 21, 2003 | 0.00 | 1.40 | 1.40 | No | N.D. | | | |
| Curtis 6E54 | Jan. 16, 2003 | 163.21 | 2.35 | 2.35 | Yes | 1.8 | 188,000,000 | 90 | 83 |
| DOB: | Jan. 23, 2003 | 68.91 | 2.40 | 2.40 | N/A | | | | |
| Apr. 13, 1999 | Jan. 30, 2003 | 99.23 | 2.40 | 2.40 | N/A | | | | |
| Dose | Feb. 6, 2003 | 26.78 | 2.10 | 2.10 | N/A | | | | |
| 400 μg/0.5 ml | Feb. 13, 2003 | 0.00 | 2.10 | 2.10 | N/A | | | | |
| on 1/1703 | Feb. 20, 2003 | 0.00 | 2.00 | 1.90 | Yes | 0.25 | 3,220,000 | <10 | 4.5 |
| | Mar. 6, 2003 | 0.00 | 1.60 | 1.60 | No | N.D. | | | |
| | Mar. 21, 2003 | 0.00 | 1.40 | 1.40 | No | N.D | | | |

*N/A, not attempted; N.D., not detected.

### REFERENCES

1. American Pet Products Manufacturers Association 1996 National Pet Owners Survey.

2. Anchel, M: Overpopulation of Cats and Dogs: Causes, Effects and Prevention. Fordham University Press, NY 1990.

3. Talwar, G P and Gaur, A: Recent Developments in Immunocontraception. Am. J. Obstet Gynecol 1987; 157: 1075-8.

4. Alexander, N J and Bialy G: Contraceptive Vaccine Development. Reprod. Fertil. Dev., 1994; 6: 273-80.

5. Ladd, A, Tsong Y Y, Walfield, A M and Thau R: Development of an Antifertility Vaccine for Pets Based on Active Immunization Against Luteinizing Hormonr Releasing Hormone. Biology of Reproduction. 1994; 51:1076-1083.

6. Chengalvala, M V, Bhatt, R A, Bhat, B M, Vernon, S K, and Lubeck, M D: Enhanced Immunogenicity of Hepatitis B Serface Antigen by Insertion of a Helper T Cell Epitope from Tetanus Toxoid. Vaccine 1999; 17: 1035-41.

7. Kaul R, Afzalpurkar A, and Gupta S K: Strategies for designing an immunocontraceptive vaccine based on zona pellucida synthetic peptides and recombinant antigen. Journal of Reproduction and Fertility 1996; 50: 127-134.

8. Mahi-Brown C A, Yanagimchi R, Hoffinan J C and Huang T T F. Fertility control in the bitch by active immunization with porcine zona pellucida: use of different adjuvants and patterns of estradiol and progesterone levels in estrus cycles. Biology of Reproduction; 1985; 32: 761-772.

9. Gorman, S P, Levy, J K, Hampton, A L, Collante, W R, Harris, A L and Brown, R G: Evaluation of a Porcine Zona Pellucida Vaccine for the Immunocontraception of Domestic Kittens. Proceedings of the Symposium on Non-Surgical Contraception of Dogs and Cats, Callaway Gardens, Ga., Apr. 19-21, 2002.

10. Fraser H M, Gunn A, Feffcoate S L, and Holland D T. Effect of active immunization to luteinizing hormone releasing hormone on serum and pituitary gonadotrophins, testes and accessory sex organs in the male rat. Journal of Endocrinology. 1974; 63: 399-406.

11. Clarke I J, Fraser H M and McNeilly A S. Active immunization of ewes against luteinizing hormone releasing hormone and its effects on ovulation and gonadotrophin, prolactin and ovarian steroid secretion. Journal of Endocrinology. 1978; 78: 39-47.

12. Silversides, D W, Murphy B D, Mapletoft R J, Misra V, and Allen A F. Agents and procedures for provoking an immune response to GnRH and immunosterilizing mammals. U.S. Pat. No. 4,975,420, 1990.

13. Jeffcoate S L, Holland D T, Fraser H M and Gunn A. Preparation and specificity of antibodies to the decapeptide, luteinizing hormone releaseing hormone. Immunochemistry. 1974; 11: 7-77.

14. C-T Hsu, C-Y Ting, C-J Ting, T-Y Chen, C-P Lin, J Whang-Peng, and J. Hwang: Vaccination Against Gonadotropin-releasing Hormone (GnRH) Using Toxin Receptor-binding Domain-conjugated GnRH Repeats. Cancer Research 2000; 60: 3701-3705.

15. Shi, Z., Zeng, M., Yang, G., Siegel, F., Cain, L. J., Van Kampen, K. R., Elmets, C. A., and Tang, D. C. Protection against tetanus by needle-free inoculation of adenovirus-vectored nasal and epicutaneous vaccines. J. Virol. 2001; 75: 11474-11482.

16. Fallaux, F. J., Bout, A., van der Velde, I., van den Wollenberg, D. J., Hehir, K. M., Keegan, J., Auger, C., Cramer, S. J., van Ormondt, H., van der Eb, A. J., Valerio, D., and Hoeben, R. C. New helper cells and matched early region 1-deleted adenovirus vectors prevent generation of replication-competent adenoviruses. Hum. Gene Ther. 1998; 9:1909-1917.

17. Makoff, A. J., Oxer, M. D., Romanos, M. A., Fairweather, N. F., and Ballantine, S. Expression of tetanus toxin fragment C in E. Coli: High level expression by removing rare codons. Nucleic Acids Research 1989; 17: 10191.

18. Lo, R. Y., Shewen, P. E., Strathdee, C. A., and Greer, C. N. Cloning and expression of the leukotoxin gene of Pasteurella haemolytica Al in Escherichia coli K-12. Infect. Immun. 1985; 50: 667-671.

19. Wernette, C. M, Smith, B. F., Barksdale, Z. L., Hecker, R., and Baker, H. J. CpG oligodeoxynucleotides stimulate canine and feline immune cell proliferation. Vet. Immunol. Immunopathol. 2002; 84, 223-236.

1. An immunogenic composition comprising
a) GnRH multimer and an antigenic carrier; or,
b) a recombinant vector for expression in vivo in, or uptake by, a host cell, wherein the recombinant vector contains a nucleic acid molecule encoding a GnRH multimer and optionally an antigenic carrier.

2. The immunogenic composition of claim 1 wherein the antigenic carrier is a subfragment of a bacterial toxin.

3. The immunogenic composition of claim 1 wherein the antigenic carrier is tetanus toxin C fragment.

4. The immunogenic composition of claim 1 wherein the GnRH multimer and the antigenic carrier are translationally conjugated.

5. The immunogenic composition of claim 1 further comprising one or more adjuvants.

6. The immunogenic composition of claim 5 wherein the adjuvant is an oligonucleotide containing CpG sequences.

7. (canceled)

8. The immunogenic composition of claim 1 wherein the antigenic carrier is present and is a subfragment of a bacterial toxin.

9. The immunogenic composition of claim 1 wherein the antigenic carrier is present and is tetanus toxin C fragment.

10. The immunogenic composition of claim 1 wherein the antigenic carrier is present and is translationally conjugated to the GnRH multimer.

11. The immunogenic composition of claim 1 wherein the recombinant vector is viral.

12. The immunogenic composition of claim 1 wherein the recombinant vector is adenoviral.

13. The immunogenic composition of claim 1 wherein the recombinant vector is bacterial.

14. The immunogenic composition of claim 1 further comprising one or more adjuvants.

15. The immunogenic composition of claim 14 wherein the adjuvant is an oligonucleotide containing CpG sequences.

16. A method for treating androgen-dependent prostate tumors in a human comprising administering the immunogenic composition of claim 1 to the human.

17. A method for treating GnRH-sensitive ovarian tumors in a human comprising administering the immunogenic composition of claim 1 to the human.

18. A method for modifying sexual physiology or behavior, or both, in an animal comprising administering the immunogenic composition of claim 1 to the animal.

19. The method of claim 18 wherein the immunogenic composition is encapsulated.

20. The method of claim 18 wherein the immunogenic composition is administered orally, topically, intradermally, mucosally, or by injection into body tissue.

21. The method of claim 18 wherein the animal is a vertebrate.

22. The method of claim 18 wherein the animal is a mammal.

23. The method of claim 18 wherein the animal is a companion animal.

24. The method of claim 18 wherein the animal is feline, canine, porcine, ovine, bovine, avian, or murine.

25. Antibodies elicited by the immunogenic composition of claim 1.

26. The antibodies of claim 25 wherein the antibodies are polyclonal.

27. The antibodies of claim 25 wherein the antibodies are monoclonal.

28. A method for quantifying GnRH comprising contacting the antibodies of claim 25 with GnRH.

29. A method for modifying sexual physiology or behavior, or both, in an animal comprising administering the antibodies of claim 25 to the animal.

30. A method for the production of meat having improved organoleptic qualities comprising administering the immunogenic composition of claim 1 or antibodies elicited by the immunogenic composition of claim 1 to male cattle, sheep, chicken or pigs.

31. The method of claim 30 wherein the immunogenic composition is administered more than once.

32. The method of claim 30 wherein the immunogenic composition is administered before the fattening phase of the animal to induce a primary low-intensity immune response that allows development of male traits, and wherein the immunogenic composition is subsequently administered shortly before slaughter to interdict the action of androgenic and non-androgenic steroids.


SEQUENCE LISTING (8 sequences)

SEQ ID NO: 1 - DNA, 30 bp, Unknown Organism
  CDS (1)..(30)
  Description of Unknown Organism GnRH nucleotide sequence
gaacattggtcatatggactacggccggga

SEQ ID NO: 2 - protein, 10 aa, Unknown Organism
  Description of Unknown Organism GnRH peptide sequence
EHWSYGLRPG

SEQ ID NO: 3 - DNA, 120 bp, Artificial Sequence
  Description of Artificial Sequence Synthetic oligonucleotide
tgtgtggaattgtgagcggataacaatttcacacaggaggaaaaaaccatggtcgacttaatacgactcactatagggccttatgggcccggtacccggatcctcgagagcttagccgtc

SEQ ID NO: 4 - protein, 4 aa, Artificial Sequence
  Description of Artificial Sequence Synthetic peptide
SLAV

SEQ ID NO: 5 - DNA, 117 bp, Artificial Sequence
  Description of Artificial Sequence Synthetic oligonucleotide
gttctgcagcgtcgcgactgggaaaacccgggcgttacccaattgaatcgattagctgcgcatcccccattcgctagctggcggaattccgaagaggcgcgcaccgataggccttcc

SEQ ID NO: 6 - protein, 39 aa, Artificial Sequence
  Description of Artificial Sequence Synthetic peptide
VLQRRDWENPGVTQLNRLAAHPPFASWRNSEEARTDRPS

SEQ ID NO: 7 - DNA, 138 bp, Artificial Sequence
  Description of Artificial Sequence Synthetic oligonucleotide
caacagttgagatctttaaatggcgaatggcggtaagcttcgaacgcgtatgcatgagctcttaattaactccggatctagagcccgcctaatgagcgggcttttttttcttaagtaaattgtaagcgttaatatttt

SEQ ID NO: 8 - protein, 11 aa, Artificial Sequence
  Description of Artificial Sequence Synthetic peptide
QQLRSLNGEWR